(19) United States
(12) Patent Application Publication
(10) Pub. No.: US 2006/0207613 A1
(43) Pub. Date: Sep. 21, 2006

(54) Device for preventing finger sucking, particularly for babies

(75) Inventor: Manuel Ibanez Gallego, Barcelona (ES)

Correspondence Address: WOLF BLOCK SCHORR AND SOLIS-COHEN LLP, 250 PARK AVENUE, NEW YORK, NY, 10177, US

(73) Assignee: Cranc Solutions, S.L., Barcelona (ES)

(21) Appl. No.: 11/355,347

(22) Filed: Feb. 16, 2006

(30) Foreign Application Priority Data
Mar. 4, 2005 (ES) 200500493

(51) Int. Cl. A61F 5/37

(52) U.S. Cl. 128/878

(57) ABSTRACT

It includes an annular body (2), designed for placing around the baby's wrist (3), and presenting a thickness (G) in radial direction sufficient to stop the movement of the hand towards the mouth due to the outer surface of said annular body (2) coming into contact with the baby's face (3). The annular body (2) is manufactured from a flexible and light material to prevent the baby's face being injured, and also it is very comfortable due to being worn like a bracelet. The device is structurally very simple thus its cost and production time is substantially low.

The present invention relates to a device for preventing finger-sucking, particularly for babies.

### BACKGROUND OF THE INVENTION

Involuntary finger-sucking is a well-known problem, particularly as it relates to babies' thumbs, so that it is desirable to eradicate this bad habit as soon as possible.

Known in the art for avoiding this problem are devices that comprise a thimble-piece or sheath that covers the finger(s)/thumb(s) whose sucking is to be stopped, said sheath being attached to the hand or wrist by means of a tape or a glove that covers part of the hand. Thus, when the user moves the finger/thumb towards the mouth, the sheath prevents the finger/thumb being sucked.

Also known are rigid devices that are placed in the elbow zone to prevent the baby from bending its arm and therefore also prevent it from bringing its finger/thumb to its mouth.

Devices such as those described are nevertheless annoying to the user, as well as very eye-catching and aesthetically displeasing.

### DESCRIPTION OF THE INVENTION

The object of the device of the present invention for preventing finger/thumb sucking, particularly for babies, is to solve the disadvantages presented by the devices known in the art, while providing a number of advantages that will be described below.

The device of the present invention for preventing finger-sucking, particularly for babies, is characterised in that it comprises an annular body, designed for placing around the baby's wrist and presenting a thickness in radial direction sufficient to stop the movement of the hand towards the mouth, due to the outer surface of said annular body coming into contact with the baby's face.

A device is thereby obtained to prevent finger-sucking, particularly for babies, which is very comfortable due to being worn like a bracelet, while it is of low cost and short production time because of being a very structurally simple device.

Advantageously, the annular body is manufactured from a flexible and light material, so as to avoid the baby hurting its face when it brings its hand to its mouth.

Preferably, the device of the invention includes means for opening and closing the annular body.

According to one embodiment of the invention, the opening and closing means include a transversal opening that allows the open ends of the annular body to be separated, thereby making it very easy to open and close.

Advantageously, the orifice of the annular body has a shape largely coinciding with the outline of the baby's wrist, so as to ensure correct attachment of the device onto the wrist and thus prevent the device from moving or being removed by the baby.

### BRIEF DESCRIPTION OF THE DRAWINGS

In order to facilitate description of the aspects outlined above some drawings are attached that show, schematically and solely by way of non-restrictive example, a practical case of embodiment of the device of the invention for preventing finger-sucking, particularly for babies, in which:

FIG. 1 is a schematic view of the device of the invention for preventing finger-sucking, fitted on the wrist of a baby;

FIG. 2 is a plan view of the device of the invention; and

FIG. 3 is a perspective view of the device of the invention.

### DESCRIPTION OF A PREFERRED EMBODIMENT

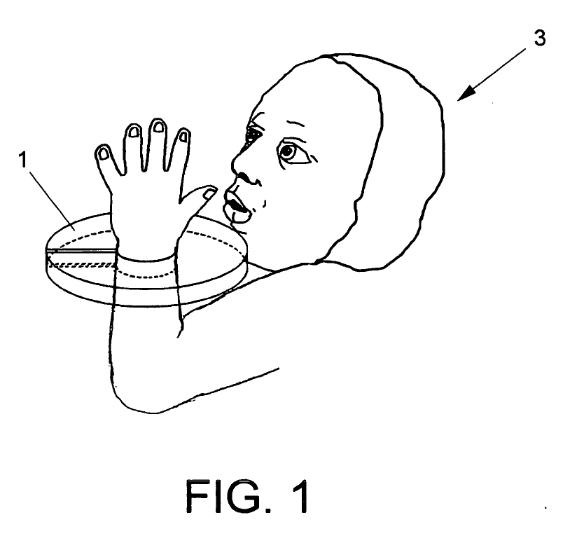

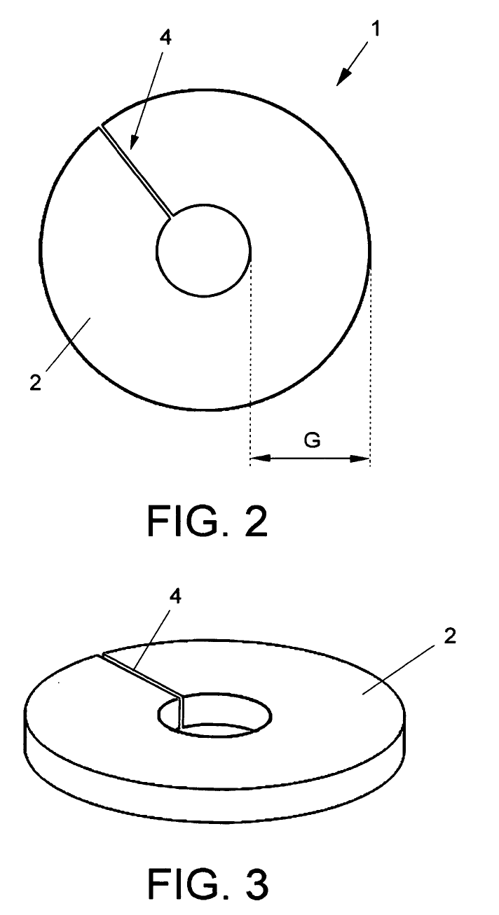

FIGS. 1 to 3 show a device 1 for preventing thumb-sucking in babies, which comprises an annular body 2 for placing around the baby's wrist 3 by way of a bracelet. Said annular body 2 has a thickness G in radial direction (see FIG. 2) appropriate for halting the movement of bringing a hand to the mouth, due to the outer surface of said annular body 2 coming into contact with the baby's face 3. Said annular body 2 is manufactured from a flexible and light material to prevent the baby's face being injured when it raises its hand to its mouth.

The device 1 also includes means for opening and closing the annular body 2, which in this embodiment are made up of a transversal opening 4 that allows the open ends of the annular body 2 to be separated easily, thanks especially to the flexibility of the material.

The central orifice of the annular body presents a shape that largely coincides with the outline of the baby's wrist 3, in order to ensure correct attachment of the device 1 on the wrist and thereby prevent the device 1 from moving or being removed by the baby 3.

1. Device (1) for preventing finger-sucking, particularly for babies, comprising an annular body (2), designed for placing around the baby's wrist (3), and presenting a thickness (G) in radial direction sufficient to stop the movement of the hand towards the mouth due to the outer surface of said annular body (2) coming into contact with the baby's face (3).

2. Device (1), according to claim 1, wherein the annular body (2) is manufactured from a flexible and light material.

3. Device (1), according to claim 1, further comprising means (4) for opening and closing the annular body (2).

4. Device (1), according to claim 3, wherein the opening and closing means include a transversal opening (4) that allows the open ends of the annular body (2) to be separated.

5. Device (1), according to claim 1, wherein the orifice of the annular body (2) has a shape largely coinciding with the outline of the baby's wrist (3).
